(19) United States
(12) Patent Application Publication
(10) Pub. No.: US 2019/0082936 A1
(43) Pub. Date: Mar. 21, 2019

(54) IMAGE PROCESSING APPARATUS

(71) Applicant: OLYMPUS CORPORATION, Tokyo (JP)

(72) Inventor: Ryuichi YAMAZAKI, Tokyo (JP)

(73) Assignee: OLYMPUS CORPORATION, Tokyo (JP)

(21) Appl. No.: 16/194,565

(22) Filed: Nov. 19, 2018

Related U.S. Application Data
(63) Continuation of application No. PCT/JP2017/021433, filed on Jun. 9, 2017.

(30) Foreign Application Priority Data
Jun. 23, 2016 (JP) 2016-124569

(51) Int. Cl. A61B 1/00; A61B 1/045; H04N 7/18; G06T 11/60; G05B 19/042

(52) U.S. Cl. CPC A61B 1/00059; A61B 1/045; H04N 7/185; A61B 1/0684; G05B 19/0423; G06T 2207/10068; G06T 11/60

(57) ABSTRACT

An image processing apparatus includes: an image signal processing circuit formed by a rewritable logic circuit and configured to perform signal processing on an image signal according to a type of the endoscope; a display image processing circuit formed by a rewritable logic circuit and configured to generate a display image signal corresponding to a display mode of a display apparatus; and a control circuit configured to control the display image processing circuit to perform configuration, control the image signal processing circuit to perform configuration according to a type of the endoscope, when replacement of the endoscope with another endoscope is detected after the configurations are performed, control the image signal processing circuit to perform reconfiguration according to a type of said another endoscope, the display image processing circuit does not perform reconfiguration when the replacement is detected, and control the display apparatus to display a display image.

### CROSS-REFERENCE TO RELATED APPLICATION

This application is a continuation of PCT International Application No. PCT/JP2017/021433 filed on Jun. 9, 2017 which claims the benefit of priority from Japanese Patent Application No. 2016-124569, filed on Jun. 23, 2016, the entire contents of which are incorporated herein by reference.

### BACKGROUND

The present disclosure relates to an image processing apparatus.

Endoscope systems for in-vivo observation of subjects have been used in the medical field. Generally, an endoscope captures in-vivo images by: insertion of an elongated and flexible insertion unit thereof into a subject, such as a patient; illumination, from a distal end of this insertion unit, with illumination light supplied by a light source device; and reception of reflected light of this illumination light by an imaging unit thereof at the distal end of the insertion unit. The in-vivo images thus captured by the imaging unit of the endoscope are displayed on a display of an endoscope system after being subjected to predetermined image processing in a processing apparatus of the endoscope system. A user, such as a medical doctor, performs observation of an organ of the subject, based on the in-vivo images displayed on the display.

In endoscopy, various endoscopes are used according to different observation purposes and observation regions. Since the content of image processing differs according to the imaging element of the endoscope in the endoscope system, different image processing circuits have been provided in the processing apparatus, or different processing apparatuses respectively corresponding to the different types of endoscopes have been individually provided. Therefore, there has been a demand for compatibility with different types of endoscopes just by a single processing apparatus having a simpler formation. & To meet this demand, an endoscope system has been proposed (see, for example, Japanese Patent Application Laid-open No. 2013-150666), in which an image processing circuit of a processing apparatus is formed by use of a field programmable gate array (FPGA), a memory is provided in each endoscope, the memory storing program data corresponding to the endoscope, the processing apparatus causes the FPGA to read the program data in an endoscope, and a logic circuit is caused to rewrite the program data, the logic circuit being able to execute image processing corresponding to an imaging element of the connected endoscope.

### SUMMARY

An image processing apparatus according to one aspect of the present disclosure performs signal processing on an image signal captured by an endoscope connected thereto, the image processing apparatus including: an image signal processing circuit that is formed by use of a rewritable logic circuit, the image signal processing circuit being configured to perform signal processing on the image signal according to a type of the endoscope; a display image processing circuit that is formed by use of a rewritable logic circuit, the display image processing circuit being configured to generate, based on a processed signal obtained by the signal processing of the image signal processing circuit, a display image signal corresponding to a display mode of a display apparatus; and a control circuit configured to control the display image processing circuit to perform configuration when the image processing apparatus is started up, control the image signal processing circuit to perform configuration according to a type of the endoscope connected to the image processing apparatus, when replacement of the endoscope with another endoscope is detected after the configurations are performed by the image signal processing circuit and the display image processing circuit, control the image signal processing circuit to perform reconfiguration according to a type of said another endoscope, wherein the display image processing circuit does not perform reconfiguration when the replacement of the endoscope with said another endoscope is detected, and control the display apparatus to display a display image based on the display image signal generated by the display image processing circuit.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic formation of an endoscope system according to an embodiment;

FIG. 2 is a block diagram illustrating a schematic formation of the endoscope system according to the embodiment;

FIG. 3 is a diagram for explanation of endoscopes that are able to be attached to a processing apparatus illustrated in FIG. 2;

FIG. 4 is a flow chart illustrating image processing performed by the processing apparatus according to the embodiment; and

FIG. 5 is a block diagram illustrating a schematic formation of an endoscope system according to a modified example of the embodiment.

### DETAILED DESCRIPTION

Hereinafter, modes for carrying out the present disclosure (hereinafter, referred to as “embodiments”) will be described. An embodiment, which is a medical endoscope system for capturing and displaying in-vivo images of subjects, such as patients, as an example of a system including an image processing apparatus according to the present disclosure, will be described. The disclosure is not limited by this embodiment. The same reference signs will each be assigned to parts that are the same, throughout the drawings.

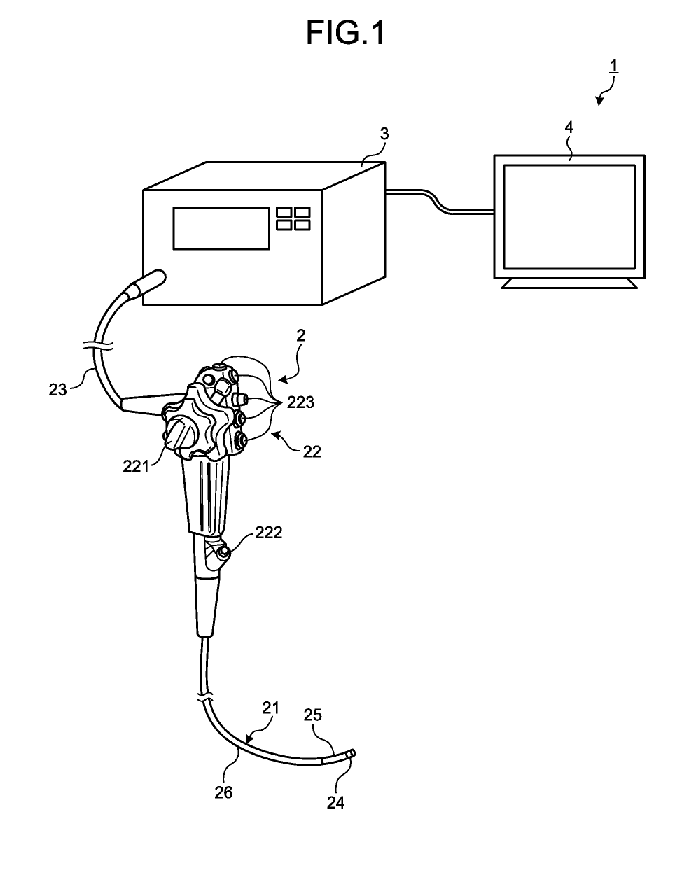

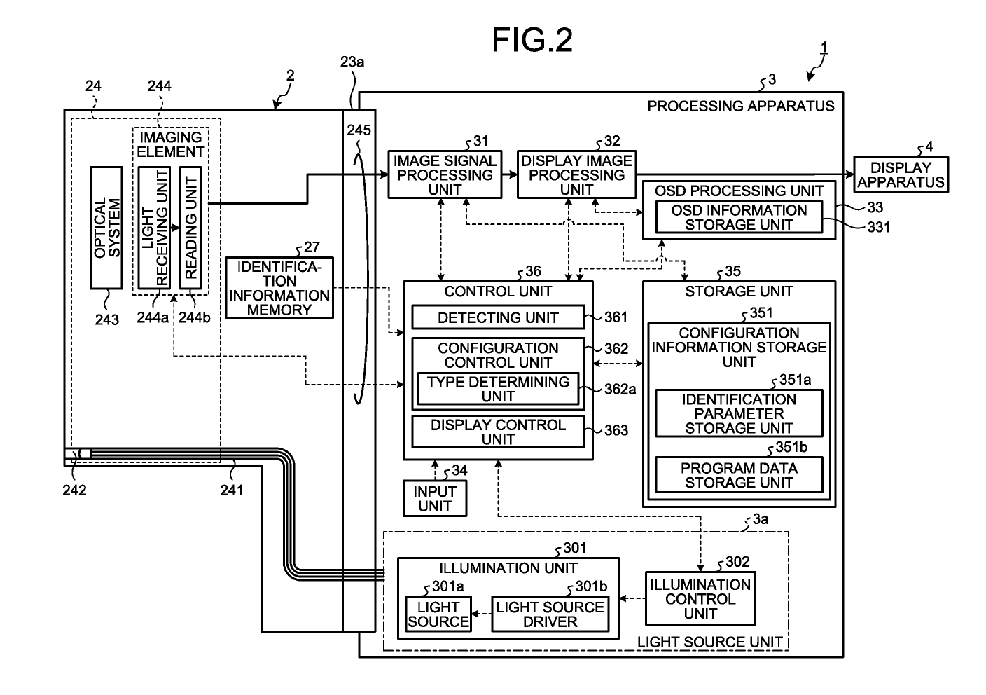

FIG. 1 is a diagram illustrating a schematic formation of an endoscope system according to the embodiment. FIG. 2 is a block diagram illustrating a schematic formation of the endoscope system according to the embodiment. In FIG. 2, solid lined arrows represent transmission of electric signals related to images, and broken lined arrows represent transmission of electric signals related to control.

An endoscope system 1 illustrated in FIG. 1 and FIG. 2 includes: an endoscope 2 for capturing in-vivo images (hereinafter, also referred to as endoscopic images) of a subject by insertion of a distal end portion thereof into the subject; a processing apparatus 3 that includes a light source unit 3a, which generates illumination light to be emitted from a distal end of the endoscope 2, that performs predetermined signal processing on image signals captured by the endoscope 2, and that integrally controls operation of the whole endoscope system 1; and a display apparatus 4 that displays thereon the endoscopic images generated through the signal processing by the processing apparatus 3.

The endoscope 2 includes: an insertion unit 21 that has flexibility, and that is elongated; an operating unit 22 that is connected to a proximal end of the insertion unit 21 and that receives input of various operation signals; and a universal cord 23 that extends in a direction different from a direction, in which the insertion unit 21 extends from the operating unit 22, and that includes various cables built therein for connection to the processing apparatus 3 (including the light source unit 3a).

The insertion unit 21 includes: a distal end portion 24 having an imaging element 244 built therein, the imaging element 244 having two-dimensionally arranged pixels that generate a signal by receiving and photoelectrically converting light; a bending portion 25 that is formed of plural bending pieces and that is freely bendable; and a flexible tube portion 26 that is connected to a proximal end of the bending portion 25, that has flexibility, and that is elongated. The insertion unit 21 is inserted into a body cavity of the subject, and captures, through the imaging element 244, an image of an object, such as a living tissue that is at a position where external light is unable to reach.

The distal end portion 24 includes: a light guide 241 that is formed by use of glass fiber, and that forms a light guiding path for light emitted by the light source unit 3a; an illumination lens 242 that is provided at a distal end of the light guide 241; an optical system 243 for condensation; and the imaging element 244 (imaging unit) that is provided at an image forming position of the optical system 243, that receives light condensed by the optical system 243, that photoelectrically converts the light into an electric signal, and that performs predetermined signal processing on the electric signal.

The optical system 243 is formed by use of one or plural lenses, and has: an optical zooming function for change of the angle of view; and a focusing function for change of the focus.

The imaging element 244 generates an electric signal (image signal) by photoelectrically converting light from the optical system 243. Specifically, the imaging element 244 includes: a light receiving unit 244a having plural pixels, which are arranged in a matrix, each of which has a photodiode that accumulates electric charge according to quantity of light and a condenser that converts an electric charge transferred from the photodiode into a voltage level, and each of which generates an electric signal by photoelectrically converting light from the optical system 243; and a reading unit 244b that sequentially reads electric signals generated by pixels arbitrarily set as targets to be read, from among the plural pixels of the light receiving unit 244a, and that outputs the read electric signals as image signals. The light receiving unit 244a includes color filters provided therein, and each pixel receives light of one of wavelength bands of red (R), green (G), and blue (B) color components. The imaging element 244 controls various operations of the distal end portion 24, according to drive signals received from the processing apparatus 3. The imaging element 244 is realized by use of, for example, a charge coupled device (CCD) image sensor, or a complementary metal oxide semiconductor (CMOS) image sensor. Further, the imaging element 244 may be a single plate image sensor; or plural image sensors of, for example, the three plate type, may be used as the imaging element 244.

The operating unit 22 includes: a bending knob 221 that bends the bending portion 25 upward, downward, leftward, and rightward; a treatment tool insertion portion 222, through which treatment tools, such as biopsy forceps, an electric knife, and an examination probe, are inserted into the body cavity of the subject; and plural switches 223 serving as an operation input unit, through which operation instruction signals are input, the operation instruction signals being for, in addition to the processing apparatus 3, a gas feeding means, a water feeding means, and a peripheral device for screen display control. A treatment tool inserted from the treatment tool insertion portion 222 comes out from an opening (not illustrated in the drawings) via a treatment tool channel (not illustrated in the drawings) of the distal end portion 24.

The universal cord 23 includes at least the light guide 241, and a cable assembly 245 that is assembled of one or plural signal lines, built therein. The cable assembly 245 includes a signal line for transmission of an image signal, a signal line for transmission of a drive signal for driving the imaging element 244, and a signal line for transmission and reception of information including specific information related to the endoscope 2 (imaging element 244). In this embodiment, transmission of an electric signal is described as being done by use of a signal line, but an optical signal may be transmitted, or a signal may be transmitted between the endoscope 2 and the processing apparatus 3 via wireless communication.

The endoscope 2 includes an identification information memory 27 for indication of identification information of the endoscope 2. The identification information memory 27 is a memory that records identification information of the endoscope 2, and that outputs the identification information of the endoscope 2 to the processing apparatus 3 by communication processing with the processing apparatus 3 when the endoscope 2 is attached to the processing apparatus 3. Or, a connection pin may be provided in a connector 23a according to a rule corresponding to the identification information of the endoscope 2, and the processing apparatus 3 may recognize the identification information of the endoscope 2, based on a state of connection between a connection pin of the processing apparatus 3 and the connection pin of the endoscope 2 when the endoscope 2 is attached to the processing apparatus 3.

Next, a formation of the processing apparatus 3 will be described. The processing apparatus 3 includes an image signal processing unit 31, a display image processing unit 32, an on-screen display (OSD) processing unit 33, an input unit 34, a storage unit 35, and a control unit 36. The image processing apparatus according to the present disclosure is formed by use of at least the image signal processing unit 31, the display image processing unit 32, the storage unit 35, and the control unit 36.

The image signal processing unit 31 receives, from the endoscope 2, an image signal, which is image data representing an endoscopic image captured by the imaging element 244. When the image signal processing unit 31 receives an analog image signal from the endoscope 2, the image signal processing unit 31 generates a digital image signal by performing A/D conversion on the analog image signal. When the image signal processing unit 31 receives an image signal as an optical signal from the endoscope 2, the image signal processing unit 31 generates a digital image signal by performing photoelectric conversion on the image signal.

The image signal processing unit 31 performs: preprocessing, such as pixel defect correction, optical correction, color correction, and optical black subtraction, on an image signal input from the endoscope 2; and signal processing, such as noise reduction, white balance adjustment, and interpolation processing, and commonalization processing of adjusting the RGB brightness to suit a preset format, on a signal generated by the preprocessing. In the pixel defect correction, a pixel value is given to a defective pixel, based on pixel values of pixels surrounding the defective pixel. In the optical correction, optical distortion of the lens is corrected. In the color correction, color temperature and color deviation are corrected. The image signal processing unit 31 generates a processed signal including a corrected image generated by the signal processing described above. The image signal processing unit 31 inputs the processed signal generated, to the display image processing unit 32.

The display image processing unit 32 performs signal processing on a signal input from the image signal processing unit 31 to generate a display image signal corresponding to a display mode of the display apparatus 4. Specifically, the display image processing unit 32 generates a display image signal, by performing zooming processing, enhancement processing, or compression processing, on an image signal. The display image processing unit 32 generates a display image by fitting an endoscopic image according to the processed signal input from the image signal processing unit 31, into a composite image (described later) input from the OSD processing unit 33 and having textual information related to the endoscopic image superimposed thereon. The display image processing unit 32 transmits a display image signal including the generated display image, to the display apparatus 4.

The image signal processing unit 31 and the display image processing unit 32 read program data input based on control by a configuration control unit 362 described later, and perform rewrite (reconfiguration) of logic circuits; through use of field programmable gate arrays (FPGAs) that are programmable logic devices with processing contents that are rewritable according to configurations. The display image processing unit 32 may be formed by use of a special-purpose processor, such as an arithmetic circuit that executes specific functions, like an application specific integrated circuit (ASIC).

The OSD processing unit 33 performs so-called on-screen display (OSD) processing, which is composition processing of generating a composite image having textual information superimposed onto a background image, for example, a black background, the background image having an area where an endoscopic image generated by the display image processing unit 32 is to be fitted in. The textual information is information indicating patient information, device information, and examination information. The OSD processing unit 33 generates textual information related to device information according to the type of the endoscope 2 connected and to imaging conditions, and forms a composite image by superimposing the textual information onto a background image.

The OSD processing unit 33 includes an OSD information storage unit 331 that stores information related to the above described OSD processing, for example, information related to the background image and to the position where the textual information is superimposed. The OSD information storage unit 331 is realized by use of a read only memory (ROM) or a random access memory (RAM).

The input unit 34 is realized by use of any of a keyboard, a mouse, switches, and a touch panel, and receives input of various signals, such as operation instruction signals for instruction for operation of the endoscope system 1. The input unit 34 may include: the switches provided in the operating unit 22; or a portable terminal, such as an external tablet computer.

The storage unit 35 stores various programs for operating the endoscope system 1, and data including various parameters needed for the operation of the endoscope system 1. The storage unit 35 also stores identification information of the processing apparatus 3. This identification information includes specific information (ID), the model year, and specification information, of the processing apparatus 3.

Further, the storage unit 35 stores various programs including an image acquisition processing program for the processing apparatus 3 to execute an image acquisition processing method. The various programs may be recorded in a computer readable recording medium, such as a hard disk, a flash memory, a CD-ROM, a DVD-ROM, or a flexible disk, and widely distributed. The various programs described above may be obtained by being downloaded via a communication network. The communication network referred to herein is realized by, for example, an existing public network, a local area network (LAN), or a wide area network (WAN), and may be wired or wireless.

Further, the storage unit 35 includes a configuration information storage unit 351 that stores configuration information according to the type of the endoscope 2 connected. The configuration information storage unit 351 includes: an identification parameter storage unit 351a that stores identification parameters for determination of, based on the identification information obtained from the endoscope 2, the type of the endoscope connected; and a program data storage unit 351b that stores plural sets of program data according to contents of image processing respectively corresponding to the imaging elements of the plural endoscopes to be attached to the processing apparatus 3.

The storage unit 35 formed as described above is realized by use of: a ROM having the various programs installed therein beforehand; and a RAM or a hard disk storing arithmetic operation parameters and data for processing.

The control unit 36 is formed by use of a general-purpose processor, such as a central processing unit (CPU), or a special-purpose processor, such as an arithmetic circuit that executes specific functions, like an ASIC, and the control unit 36 controls driving of components including the imaging element 244 and the light source unit 3a, and controls input and output of information from and to these components. The control unit 36 refers to control information data (for example, readout timing) for imaging control stored in the storage unit 35, and transmits the control information data as a drive signal to the imaging element 244 via a predetermined signal line included in the cable assembly 245.

The control unit 36 includes: a detecting unit 361 that detects connection of the endoscope 2; a configuration control unit 362 that controls configuration in the image signal processing unit 31 and the display image processing unit 32; and a display control unit 363 that performs control of causing the display apparatus 4 to display thereon an image according to a display image signal generated by the display image processing unit 32.

The detecting unit 361 detects connection between the endoscope 2 and the processing apparatus 3 by detecting: electric conduction between the endoscope 2 connected and the processing apparatus 3; or depression or arrangement of connection detecting pins.

The configuration control unit 362 includes a type determining unit 362a that determines the type of the endoscope 2 connected, by obtaining the identification information from the endoscope 2, and comparing the identification information with the identification parameters stored in the identification parameter storage unit 351a.

Next, a formation of the light source unit 3a will be described. The light source unit 3a includes an illumination unit 301 and an illumination control unit 302. Under control by the illumination control unit 302, the illumination unit 301 irradiates the object (subject) with illumination light of different exposure values that are sequentially switched over to one another. The illumination unit 301 includes a light source 301a and a light source driver 301b.

The light source 301a is formed by use of an LED light source that emits white light and one or plural lenses, and emits light (illumination light) by the LED light source being driven. The illumination light emitted by the light source 301a is output to the object from a distal end of the distal end portion 24 via the light guide 241. The light source 301a is realized by use of any of an LED light source, a laser light source, a xenon lamp, and a halogen lamp.

The light source driver 301b causes the light source 301a to emit illumination light by supplying electric current to the light source 301a, under control by the illumination control unit 302.

Based on a control signal (light control signal) from the control unit 36, the illumination control unit 302 controls the amount of electric power to be supplied to the light source 301a and controls drive timing of the light source 301a.

The display apparatus 4 displays thereon a display image corresponding to an image signal received from the processing apparatus 3 (display image processing unit 32) via a video cable. The display apparatus 4 is formed by use of a liquid crystal or organic electroluminescence (EL) monitor.

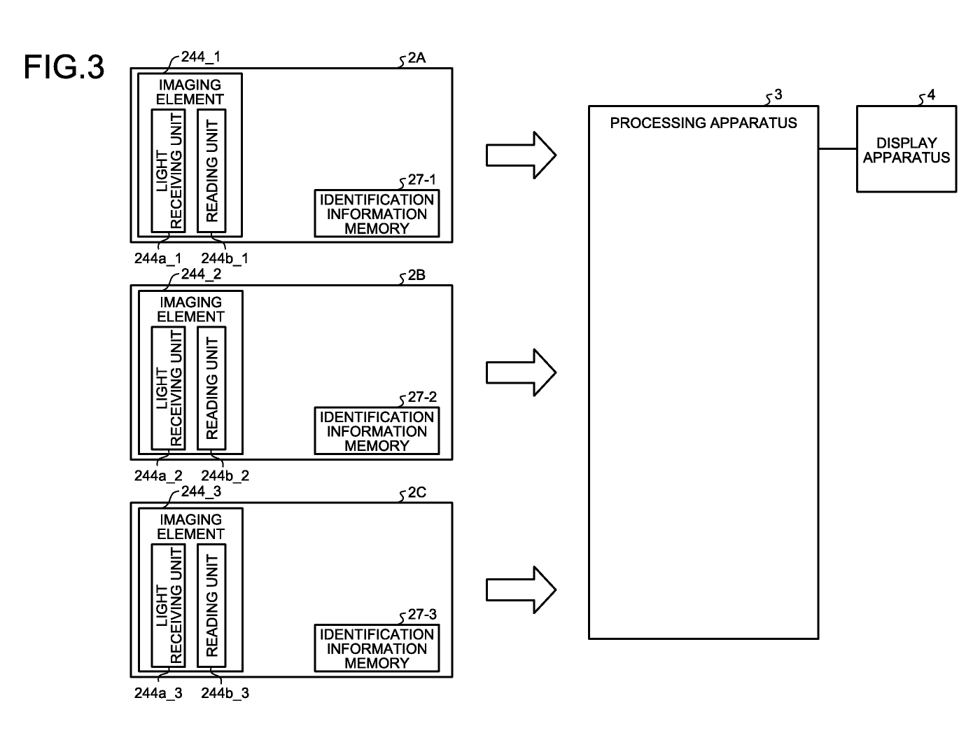

In the endoscope system 1, different types of endoscopes 2 are able to be connected to the processing apparatus 3. FIG. 3 is a diagram for explanation of endoscopes that are able to be attached to the processing apparatus illustrated in FIG. 2. For example, any one of endoscopes 2A to 2C of different types is connected to the processing apparatus 3, as illustrated in FIG. 3. The endoscopes 2A to 2C respectively include: imaging elements 244_1 to 244_3 of types different from one another; and identification information memories 27_1 to 27_3 storing identification information for identification of these endoscopes. The imaging elements 244_1 to 244_3 respectively include light receiving units 244a_1 to 244a_3 and reading units 244b_1 to 244b_3.

When the endoscope 2A illustrated in FIG. 3 is attached, the configuration control unit 362 causes the image signal processing unit 31 to read program data corresponding to the content of signal processing performed according to the imaging element 244_1 and to perform configuration. Thereby, the image signal processing unit 31 is now able to execute image processing on an image signal output by the endoscope 2A. Further, when the endoscope 2B is attached, the configuration control unit 362 causes the image signal processing unit 31 to read program data corresponding to the content of signal processing performed according to the imaging element 244_2, and to perform configuration, thereby enabling the image signal processing unit 31 to execute image processing on an image signal output by the endoscope 2B. When the endoscope 2C is attached, the configuration control unit 362 causes the image signal processing unit 31 to read program data corresponding to the content of signal processing performed according to the imaging element 244_3, and to perform configuration, thereby enabling the image signal processing unit 31 to execute image processing on an image signal output by the endoscope 2C. Therefore, any of the endoscopes 2A to 2C, for which the contents of image processing differ from one another, is enabled to be attached to the processing apparatus 3.

As described above, in this endoscope system 1, by the program data storage unit 351b of the processing apparatus 3 storing the plural sets of program data corresponding to the contents of image processing respectively corresponding to the imaging elements of the plural endoscopes to be attached to the processing apparatus 3, not matter which one of the endoscopes is attached, the image signal processing unit 31 is able to be caused to reconstruct a logic circuit according to the image processing corresponding to the imaging element of the attached endoscope.

FIG. 3 illustrates an example where three types of endoscopes 2A to 2C are attachable, but, of course, the embodiment is not limited to this example. According to the embodiment, the number of types of endoscopes 2 to be attached is plural, and sets of program data respectively corresponding to contents of image processing corresponding to imaging elements of these endoscopes are stored beforehand in the program data storage unit 351b.

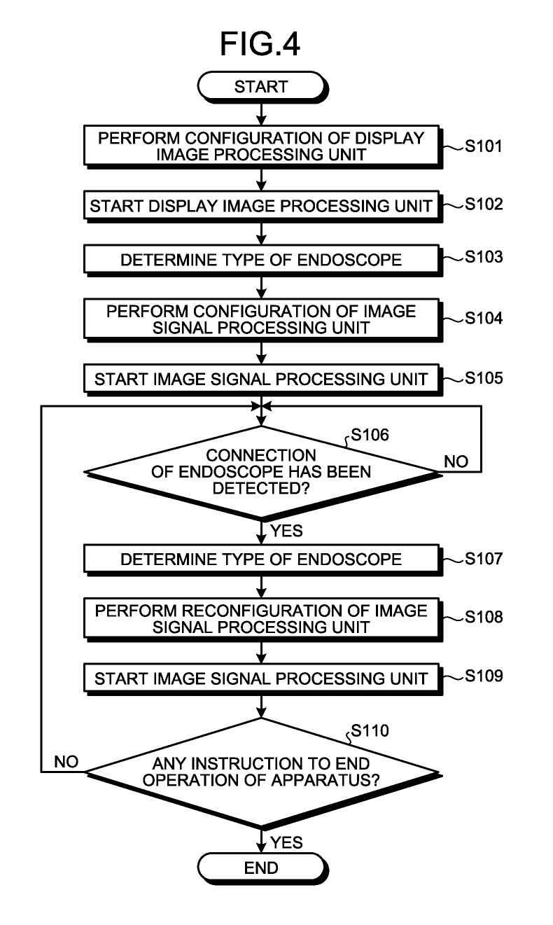

Next, image processing performed by the endoscope system 1 will be described. FIG. 4 is a flow chart illustrating image processing performed by the processing apparatus according to the embodiment. Hereinafter, description will be made on the assumption that each unit operates under control by the control unit 36. Further, the flow chart illustrated in FIG. 4 will be described on the assumption that power is supplied to the processing apparatus 3 after, for example, the endoscope 2A illustrated in FIG. 3 has been connected.

Firstly, when power is supplied to the processing apparatus 3, the configuration control unit 362 performs configuration of the display image processing unit 32 (Step S101). The configuration control unit 362 causes the display image processing unit 32 to read program data stored in the program data storage unit 351b and to perform configuration. When the display image processing unit 32 is formed of an ASIC, Step S101 may be not performed.

At Step S102 subsequent to Step S101, the display image processing unit 32 that has been configured is started. Thereby, a composite image, which has been generated by the OSD processing unit 33 and includes a blank area, for example, a blacked out area, where an endoscopic image acquired by the endoscope 2 is to be displayed, is generated as a display image. This display image is able to be displayed on the display apparatus 4, under control by the display control unit 363. In this display image, information related to the endoscope 2A that has been connected may be displayed as textual information.

At Step S103 subsequent to Step S102, the type determining unit 362a performs determination of the type of the endoscope 2A that has been connected to the processing apparatus 3. The type determining unit 362a determines the type of the endoscope 2A connected, by obtaining the identification information from the endoscope 2A, and comparing the identification information with the identification parameters stored in the identification parameter storage unit 351a.

At Step S104 subsequent to Step S103, the configuration control unit 362 performs configuration of the image signal processing unit 31. The configuration control unit 362 causes the image signal processing unit 31 to read the program data corresponding to the content of signal processing performed according to the imaging element 244_1 of the endoscope 2A, and to perform configuration.

At Step S105 subsequent to Step S104, the image signal processing unit 31 that has been configured is started. After the start, a display image is enabled to be displayed on the display apparatus 4 under control by the display control unit 363, the display image being a composite image having image information including an endoscopic image based on an image signal acquired from the endoscope 2A, and textual information related to the image information, the image information and the textual information having been superimposed onto each other.

Thereafter, the detecting unit 361 performs detection of connection of any endoscope (Step S106). Through this detection by the detecting unit 361, whether or not the endoscope 2A connected to the processing apparatus 3 is replaced with another one is determined. When the detecting unit 361 has not detected the replacement of the endoscope 2A (Step S106: No), the detecting unit 361 repeats the detection for connection. On the contrary, when the detecting unit 361 has detected the replacement of the endoscope 2A (Step S106: Yes), the flow proceeds to Step S107. Herein, description will be made on the assumption that the endoscope 2A is replaced with, for example, the endoscope 2B.

At Step S107, the type determining unit 362a performs determination of the type of the endoscope 2B that has been connected to the processing apparatus 3. The type determining unit 362a determines the type of the endoscope 2B connected, by obtaining the identification information from the endoscope 2B, and comparing the identification information with the identification parameters stored in the identification parameter storage unit 351a.

At Step S108 subsequent to Step S107, the configuration control unit 362 performs reconfiguration of the image signal processing unit 31. The configuration control unit 362 causes the image signal processing unit 31 to read the program data corresponding to the content of signal processing performed according to the imaging element 244_2 of the endoscope 2B, and to perform reconfiguration.

When, at Step S106, the endoscope 2A is simply detached and attached, at Step S108, the configuration control unit 362 may decide not to perform configuration of the image signal processing unit 31.

At Step S109 subsequent to Step S108, the image signal processing unit 31 that has been configured is started. After the start, a display image is able to be displayed on the display apparatus 4, the display image being a composite image having image information including an endoscopic image based on an image signal acquired from the endoscope 2B, and textual information related to the image information, the image information and the textual information having been superimposed onto each other.

At Step S110 subsequent to Step S109, the control unit 36 determines whether or not there is an instruction to end the operation of the processing apparatus 3. When the control unit 36 determines, for example, that input of an instruction to end the operation of the processing apparatus 3 has not been received through the input unit 34 (Step S110: No), the control unit 36 returns to Step S106 and repeats the above described processing; and when the control unit 36 determines that input of an instruction to end the operation of the processing apparatus 3 has been received through the input unit 34 (Step S110: Yes), the control unit 36 ends the above described configuration.

According to the above described embodiment, when an endoscope is connected to the processing apparatus 3 and reconfiguration is performed, since, under control by the configuration control unit 362, program data executed according to an imaging element of the endoscope connected are configured and program data for a display image are not configured, the time needed for configuration is able to be reduced.

Further, according to the embodiment, upon startup, configuration and start of the display image processing unit 32 are performed before configuration and start of the image signal processing unit 31; and when replacement of endoscopes is detected, program data executed according to an imaging element of the endoscope are reconfigured, and program data for a display image are not reconfigured. Thereby, even if the image signal processing unit 31 is under reconfiguration, a display image of textual information is able to be displayed by the display apparatus 4 on a display.

### Modified Example of Embodiment

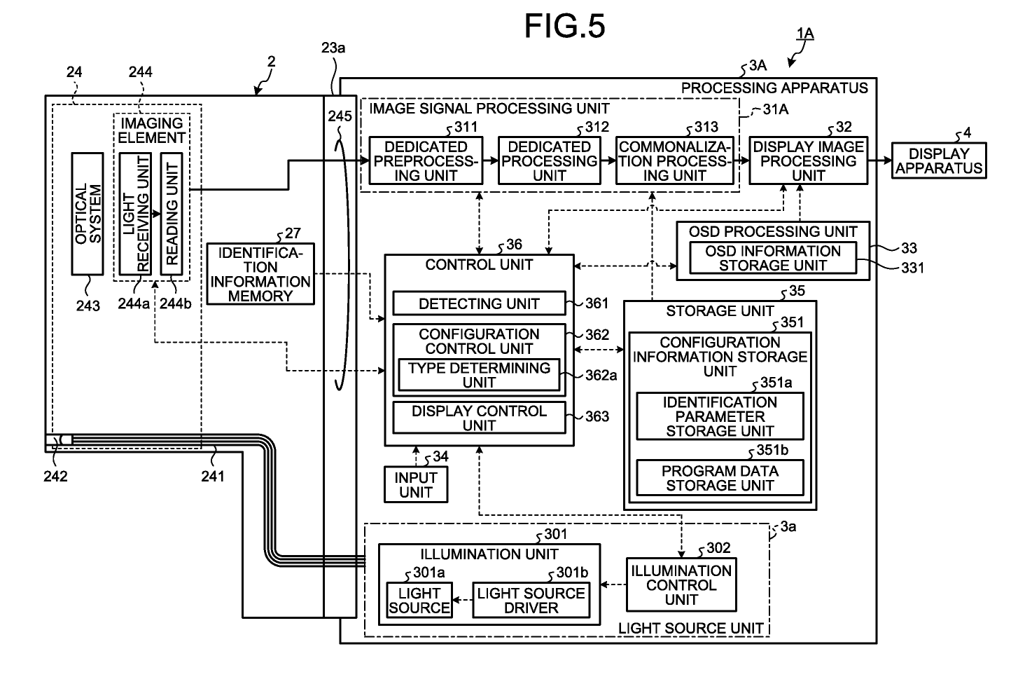

In this modified example, an image signal processing unit that performs signal processing according to an imaging element included in an endoscope is formed of plural FPGAs, and program data corresponding to each of contents of signal processing thereof are reconfigured. FIG. 5 is a block diagram illustrating a schematic formation of an endoscope system according to the modified example of the embodiment. In FIG. 5, solid lined arrows represent transmission of electric signals related to images, and broken lined arrows represent transmission of electric signals related to control.

An endoscope system 1A according to the modified example includes: the endoscope 2 for capturing in-vivo endoscopic images of a subject by insertion of the distal end portion thereof into the subject; a processing apparatus 3A that includes the light source unit 3a, which generates illumination light to be emitted from the distal end of the endoscope 2, that performs predetermined signal processing on image signals captured by the endoscope 2, and that integrally controls operation of the whole endoscope system 1A; and the display apparatus 4 that displays thereon the endoscopic images generated through the signal processing by the processing apparatus 3A. That is, the endoscope system 1A includes the processing apparatus 3A, instead of the above described processing apparatus 3 of the endoscope system 1.

The processing apparatus 3A includes an image signal processing unit 31A, the display image processing unit 32, the OSD processing unit 33, the input unit 34, the storage unit 35, and the control unit 36.

The image signal processing unit 31A receives, from the endoscope 2, an image signal, which is image data representing an endoscopic image captured by the imaging element 244. The image signal processing unit 31A includes: a dedicated preprocessing unit 311 that performs preprocessing, such as pixel defect correction, optical correction, color correction, and optical black subtraction, according to the imaging element, on an image signal input from the endoscope 2; a dedicated processing unit 312 that performs signal processing, such as noise reduction, white balance adjustment, and interpolation processing, according to an imaging element included in an endoscope connected; and a commonalization processing unit 313 that performs commonalization processing of adjusting the RGB brightness to suit a preset format. The image signal processing unit 31A inputs a processed signal generated through the commonalization processing by the commonalization processing unit 313, to the display image processing unit 32.

The dedicated preprocessing unit 311, the dedicated processing unit 312, and the commonalization processing unit 313: are formed by use of FPGAs; read program data input based on control by the configuration control unit 362; and rewrite (reconfigure) the logic circuits.

In this modified example too, configuration is performed according to the flow chart illustrated in FIG. 4. In this modified example, at Steps S104 and S108, configuration of the dedicated preprocessing unit 311, the dedicated processing unit 312, and the commonalization processing unit 313 is performed. The image signal processing unit 31A may thus be segmented into plural units, and configuration of these units may be performed, like in this modified example.

According to this modified example, when configuration is performed, when program data are common, configuration of any corresponding block may be not performed. For example, when program data of the commonalization processing unit 313 are common to endoscopes that are able to be connected, configuration by the commonalization processing unit 313 may be not performed. Thereby, the time needed for configuration is able to be reduced, and processing load is thus able to be reduced.

In the above described embodiment and modified example, by partial configuration of a single FPGA, configuration of a part of the image signal processing unit 31 or 31A may be carried out.

According to the above description of the embodiment and modified example, the configuration and start of the display image processing unit 32 are performed before the configuration and start of the image signal processing unit 31 or 31A, but the configuration and start of the image signal processing unit 31 or 31A may be performed before the configuration and start of the display image processing unit 32.

Further, according to the above description of the embodiment, the configuration information storage unit 351 is provided in the processing apparatus 3: but identification data of the endoscope 2 and program data related to configuration may be stored in an external storage device, and the processing apparatus 3 may obtain the information from this external storage device; or the configuration information storage unit 351 may be provided in the endoscope.

Further, according to the above description of the embodiment, the processing apparatus 3 generates a processed signal including an image added with RGB color components; but the processing apparatus 3 may generate a processed signal having a YCbCr color space including a luminance (Y) component and chrominance components based on the YCbCr color space, or may generate a processed signal having divided components of color and luminance by use of an HSV color space formed of three components that are hue, saturation or chroma, and value or lightness or brightness, or an L*a*b* color space using a three dimensional space.

Further, according to the above description of the embodiment, the simultaneous illumination/imaging system, in which white illumination light including RGB color components is emitted from the light source unit 3a and the light receiving unit receives reflected light arising from the illumination light, is adopted, but a field sequential illumination/imaging system, in which the light source unit 3a sequentially emits light of the color components individually, and the light receiving unit receives light of each color component, may be adopted.

Further, according to the above description of the embodiment, the light source unit 3a is formed separately from the endoscope 2, but a light source device may be provided in the endoscope 2 by, for example, provision of a semiconductor light source at the distal end of the endoscope. Furthermore, functions of the processing apparatus 3 may be provided in the endoscope 2.

Further, according to the above description of the embodiment, the light source unit 3a is provided integrally with the processing apparatus 3, but the light source unit 3a and the processing apparatus 3 may be provided separately from each other, such that, for example, the illumination unit 301 and the illumination control unit 302 are provided outside the processing apparatus 3. Furthermore, the light source 301a may be provided at the distal end of the distal end portion 24.

Further, according to the above description of the embodiment, the endoscope system according to the present disclosure is the endoscope system 1 using the flexible endoscope 2 where targets to be observed are living tissues inside subjects, but the endoscope system according to the present disclosure is also applicable to an endoscope system using a rigid endoscope, an industrial endoscope for observation of properties of materials, a capsule type endoscope, a fiberscope, or a device having a camera head connected to an eyepiece unit of an optical endoscope, such as an optical visual tube.

The present disclosure has an effect of enabling: reduction of time needed for configuration; and display of an image on a display even during configuration.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus that performs signal processing on an image signal captured by an endoscope connected thereto, the image processing apparatus comprising:
an image signal processing circuit that is formed by use of a rewritable logic circuit, the image signal processing circuit being configured to perform signal processing on the image signal according to a type of the endoscope;
a display image processing circuit that is formed by use of a rewritable logic circuit, the display image processing circuit being configured to generate, based on a processed signal obtained by the signal processing of the image signal processing circuit, a display image signal corresponding to a display mode of a display apparatus; and
a control circuit configured to
control the display image processing circuit to perform configuration when the image processing apparatus is started up,
control the image signal processing circuit to perform configuration according to a type of the endoscope connected to the image processing apparatus,
when replacement of the endoscope with another endoscope is detected after the configurations are performed by the image signal processing circuit and the display image processing circuit, control the image signal processing circuit to perform reconfiguration according to a type of said another endoscope, wherein the display image processing circuit does not perform reconfiguration when the replacement of the endoscope with said another endoscope is detected, and
control the display apparatus to display a display image based on the display image signal generated by the display image processing circuit.

2. The image processing apparatus according to claim 1, further comprising a program data storage that stores program data according to the type of the endoscope.

3. The image processing apparatus according to claim 1, wherein the image signal processing circuit is formed by use of rewritable logic circuits that perform signal processing different from each other.

4. The image processing apparatus according to claim 1, further comprising an on-screen display processing circuit configured to perform composition processing of superimposing textual information onto a background image that includes an area where the display image signal generated by the display image processing circuit is fitted in,
wherein the display control circuit controls the display apparatus to display, as the display image, a composite image obtained by the composition processing of the on-screen display processing circuit after the display image processing circuit is started up.
